(19) United States
(12) Patent Application Publication
(10) Pub. No.: US 2024/0115269 A1
(43) Pub. Date: Apr. 11, 2024

(54) IMPLANTABLE MEDICAL DEVICE ADAPTABLE TO IRREGULAR ANATOMY

(71) Applicant: BOSTON SCIENTIFIC SCIMED, INC., MAPLE GROVE, MN (US)

(72) Inventors: Jerry Wang, Edina, MN (US); James M. Anderson, Corcoran, MN (US); Joshua Mark Inouye, Brooklyn Park, MN (US)

(73) Assignee: BOSTON SCIENTIFIC SCIMED, INC., MAPLE GROVE, MN (US)

(21) Appl. No.: 18/378,391

(22) Filed: Oct. 10, 2023

Related U.S. Application Data
(60) Provisional application No. 63/415,041, filed on Oct. 11, 2022.

(51) Int. Cl. A61B 17/12

(52) U.S. Cl. CPC A61B 17/12122; A61B 2017/00632

(57) ABSTRACT

An implantable medical device such as but not limited to a left atrial appendage closure (LAAC) device includes an expandable frame that is movable between a collapsed configuration for delivery and an expanded configuration for deployment. The expandable frame may include a plurality of articulating members that are biased into the expanded configuration. The expandable frame may include a biasing member. The LAAC device includes a membrane or covering that spans across an end of the expandable frame.

### CROSS-REFERENCE TO RELATED APPLICATIONS

This application claims the benefit of priority of U.S. Provisional Application No. 63/415,041 filed Oct. 11, 2022, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device. The implantable medical device includes an expandable frame including a peripheral element, a central element rotatably disposed relative to the peripheral element, and a plurality of articulating members. Each of the plurality of articulating members have a first end secured to the central element, a second end secured to the peripheral element and a loop portion extending therebetween. A covering is disposed relative to the plurality of articulating members. Relative rotation between the central element and the peripheral element in a first relative rotational direction causes the loop portions of each of the plurality of articulating members to extend radially outwardly beyond where the second end of each of the plurality of articulating members is secured to the peripheral element.

Alternatively or additionally, the central element may include a first disk having a first diameter.

Alternatively or additionally, the peripheral element may include a second disk having a second diameter greater than the first diameter.

Alternatively or additionally, the peripheral element may include an expandable stent structure.

Alternatively or additionally, the second end of each of the plurality of articulating members may be pivotably secured to the peripheral element.

Alternatively or additionally, the implantable medical device may further include a plurality of pins pinning the second end of each of the plurality of articulating members to the peripheral element.

Alternatively or additionally, relative rotation of the central element between the peripheral element in a second relative rotational direction, opposing the first relative rotational direction, may cause the loop portions of each of the plurality of articulating members to retract radially inwardly inside of where the second end of each of the plurality of articulating members is secured to the peripheral element.

Alternatively or additionally, the covering may extend over the plurality of articulating members.

Alternatively or additionally, the plurality of articulating members may be formed of a shape memory material.

Alternatively or additionally, the implantable medical device may include an LAAC (left atrial appendage closure) device.

Another example may be found in a left atrial appendage closure (LAAC) device. The LAAC device includes a peripheral element and a central element, where the central element and the peripheral element are adapted to rotate relative to each other. The LAAC device includes a plurality of articulating members, each of the plurality of articulating members having a first end secured to the central element, a second end pivotably secured to the peripheral element and a loop portion extending therebetween. A covering is disposed relative to the plurality of articulating members. Relative rotation between the central element and the peripheral element in a first relative rotational direction causes the loop portions of each of the plurality of articulating members to extend radially outwardly beyond a periphery of the peripheral element.

Alternatively or additionally, the covering extends over the plurality of articulating members.

Alternatively or additionally, the central element may include a first disk having a first diameter and the peripheral element may include a second disk having a second diameter greater than the first diameter.

Alternatively or additionally, the peripheral element may include an expandable stent structure.

Alternatively or additionally, the plurality of articulating members may be formed of a shape memory material.

Alternatively or additionally, the plurality of articulating members may be adapted to have a remembered shape corresponding to a maximum value for the overall diameter of the LAAC device.

Another example may be found in a left atrial appendage closure (LAAC) device. The LAAC device includes an expandable frame that is moveable between a collapsed configuration and an expanded configuration. The expandable frame includes a peripheral element, a central element rotatably disposed relative to the peripheral element, and a plurality of articulating members, each of the plurality of articulating members having a first end secured to the central element, a second end secured to the peripheral element and a loop portion extending therebetween. A covering is disposed relative to the plurality of articulating members. Rotation of the central element relative to the peripheral element causes the loop portions of each of the plurality of articulating members to extend radially outwardly beyond a periphery of the peripheral element.

Alternatively or additionally, the plurality of articulating members may be formed of a shape memory material.

Alternatively or additionally, the plurality of articulating members may be adapted to have a remembered shape corresponding to the expanded configuration of the expandable frame.

Alternatively or additionally, rotating the central member in a first direction may cause the expandable frame to move towards the expanded configuration and rotating the central member in a second direction opposing the first direction may cause the expandable frame to move towards the collapsed configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional view of an LAA (left atrial appendage);

FIGS. 2A, 2B and 2C are schematic drawings showing relative expansion of an illustrative LAAC device;

FIG. 3 is a perspective view of an illustrative disk-style expandable frame for an LAAC device, shown in a collapsed configuration;

FIG. 4 is a perspective view of the illustrative disk-style expandable frame of FIG. 3, shown in an expanded configuration;

FIG. 5 is a schematic view of an illustrative delivery system for the disk-style expandable frame of FIG. 3;

FIG. 6 is a perspective view of an illustrative stent-style expandable frame for an LAAC device, shown in a collapsed configuration;

FIG. 7 is a schematic view of the illustrative stent-style expandable frame of FIG. 6, shown in an expanded configuration; and

FIGS. 8A, 8B and 8C together provide views of an illustrative membrane material that may be used as a covering on the LAAC devices described herein.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

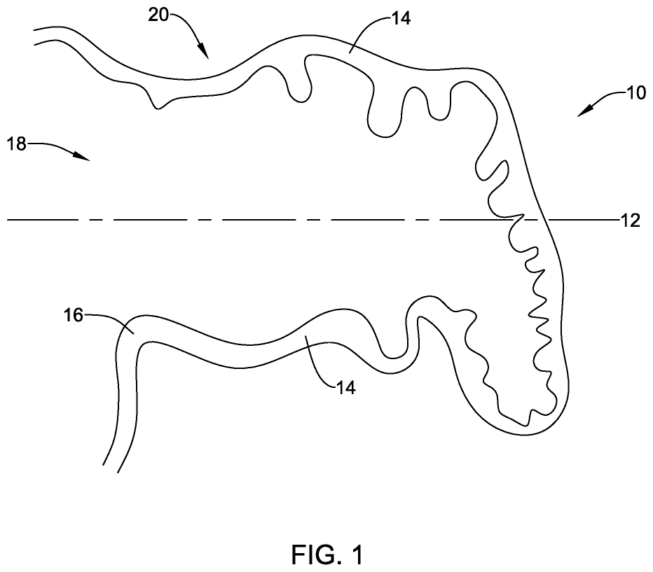

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

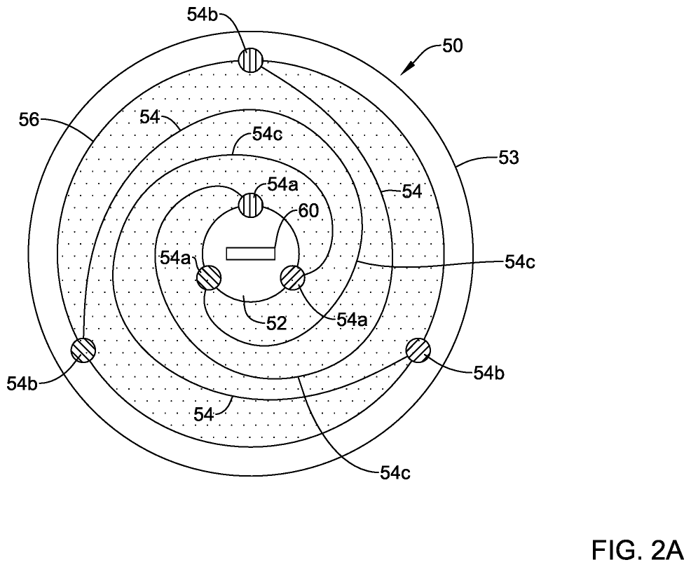

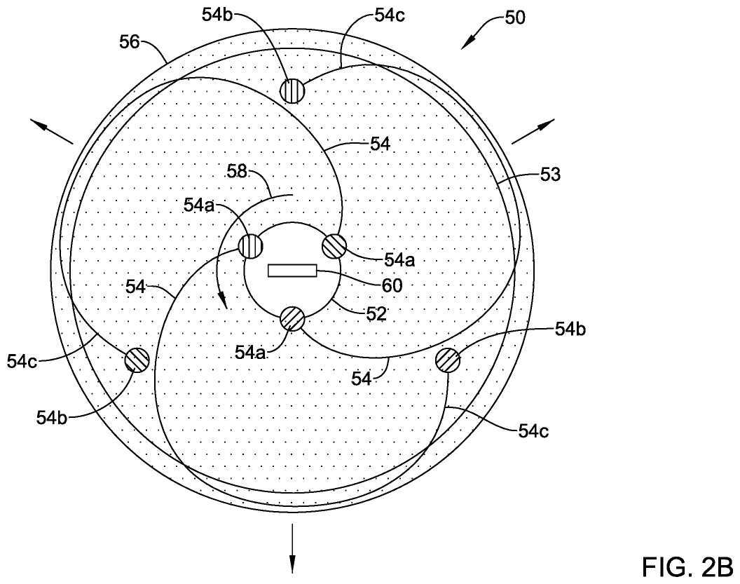

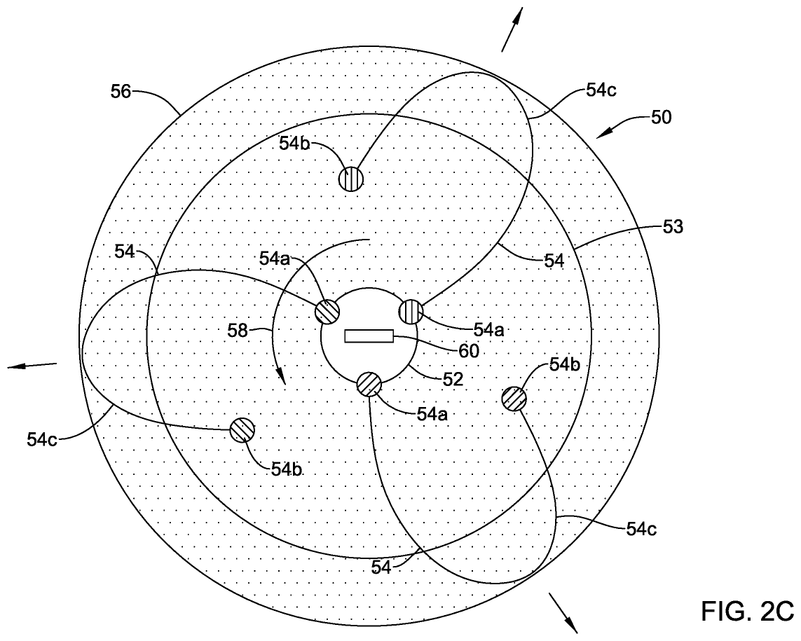

In some instances, a device known as an LAAC (left atrial appendage closure) device may be implanted within the LAA 10, such as near or within the ostium 16, in order to seal off the interior of the LAA 10 from the rest of the heart interior. FIGS. 2A, 2B and 2C are schematic views of an illustrative LAAC device 50. The illustrative LAAC device 50 has a center element 52, a peripheral element 53 and a number of articulating members 54 that extend between the center element 52 and the peripheral element 53. In some cases, each of the articulating members 54 has a first end 54a where the articulating member 54 is secured relative to the center element 52, a second end 54b that is secured relative to the peripheral element 53, and a loop portion 54c that extends between the first end 54a and the second end 54b. In some cases, the second end 54b may be considered as being secured near a periphery of the peripheral element 53.

The LAAC device 50 includes a covering 56 that extends over the articulating members 54. In some cases, the covering 56 may be secured to the second ends 54b of each of the articulating members 54. Accordingly, as the articulating members 54 are moved towards a collapsed configuration or towards an expanded configuration or a further expanded configuration, movement of the articulating members 54 will cause the covering 56 to decrease in diameter or increase in diameter in conjunction with the articulating members 54. While a total of three articulating members 54 are shown, it will be appreciated that this is merely illustrative, as the LAAC device 50 may have any number of articulating members 54.

FIG. 2A may be considered as showing the LAAC device 50 in or closest to a collapsed configuration in which the LAAC device 50 has a minimal diameter. Comparing FIG. 2B with FIG. 2A, it can be seen that the LAAC device 50 has increased in its diameter. This occurs as a result of rotating the center element 52 in a direction indicated by an arrow 58. As the center element 52 is rotated in the direction indicated by the arrow 58, and with the second end 54b of each of the articulating members 54 held in position relative to the peripheral element 53, the corresponding loop portion 54c of each of the articulating members 54 moved radially outwardly, thereby increasing the diameter of the LAAC device 50. Comparing FIG. 2C with FIG. 2B, it can be seen that the LAAC device 50 has increased further in overall diameter in response to further rotation of the center element 52 relative to the peripheral element 53.

In some cases, the center element 52 may include a feature 60 that allows engagement of the center element 52 by an elongate tool that may be used to actuate the center element 52. As shown, the feature 60 includes a straight slot that is adapted to accommodate a straight-bladed screwdriver head, or a tool including such a head. This is just an example, as the feature 60 may include a pair of orthogonal slots to accommodate a Phillips-headed tool, or perhaps a hex-shaped aperture to accommodate an elongate tool having a hex head on it. Accordingly, the overall diameter of the LAAC device 50 may be adjusted as desired or needed to accommodate the overall dimensions of the ostium 16 for a particular patient.

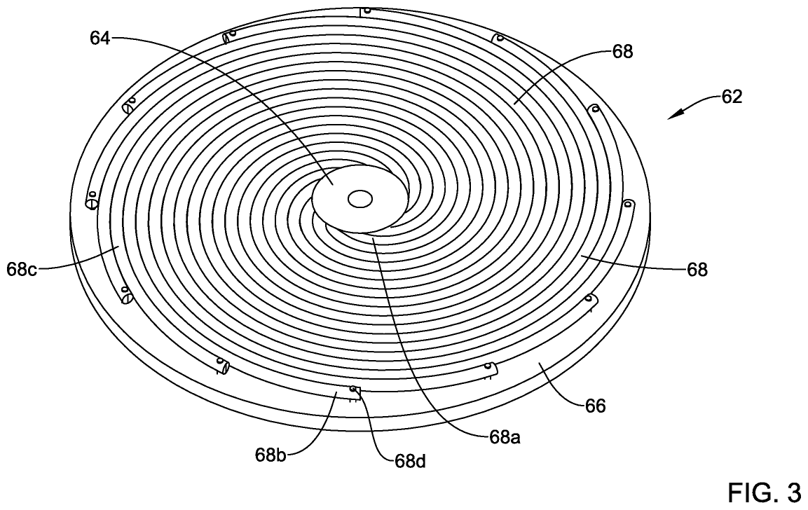

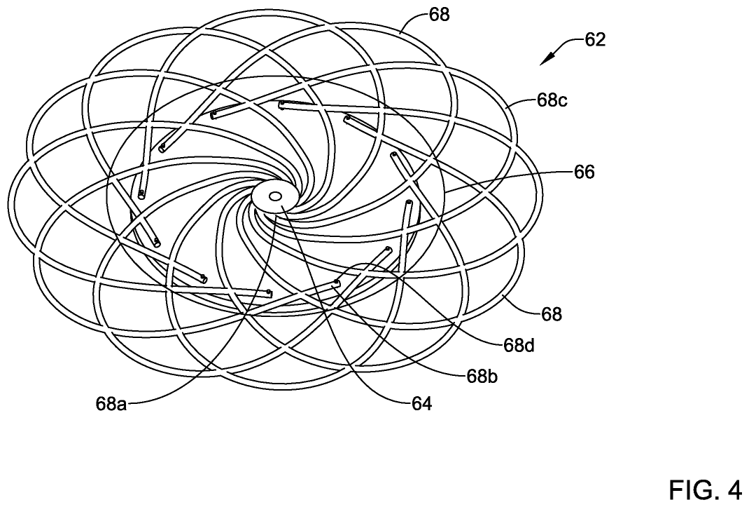

FIGS. 3 and 4 are perspective views of an illustrative expandable frame 62 that may be used in forming an LAAC device. In some cases, for example, an LAAC device could be formed simply by adding a covering (not shown) to the expandable frame 62. FIG. 3 shows the expandable frame 62 in a collapsed configuration while FIG. 4 shows the expandable frame 62 in an expanded configuration. The expandable frame 62 includes a first disk 64 and a second disk 66, with a number of articulating members 68 extending between the first disk 64 and the second disk 66. In some cases, the first disk 64 may be considered as being an example of the center element 52 and the second disk 66 may be considered as being an example of the peripheral element 53.

In some cases, each of the articulating members 68 has a first end 68a by which the articulating member 68 is secured relative to the first disk 64, and a second end 68b by which the articulating member 68 is secured relative to the second disk 66. In some cases, each of the articulating members 68 include a loop portion 68c that extends between the first end 68a and the second end 68b. In some cases, the second end 68b of each of the articulating members 68 is pivotably secured relative to the second disk 66. As an example, the second end 68b of each of the articulating members 68 may be secured to the second disk 66 via a pin 68d that extends through the second end 68b and into the second disk 66. This is just an example.

The articulating members 68 may be considered as being formed of a shape memory material that is adapted to have a remembered configuration. The articulating members 68 may be adapted to be deformed from the remembered configuration (such as collapsing the LAAC device into a delivery configuration), and to regain the remembered configuration once no longer constrained. As an example, the articulating members 68 may be formed of a shape memory metal such as Nitinol. As another example, the articulating members 68 may be formed of a shape memory polymer. The first disk 64 and the second disk 66 may be formed of any suitable materials.

In some cases, each of the articulating members 68 may have a circular or substantially circular cross-sectional shape. The articulating members 68 may be formed of a suitable wire, for example. In some cases, the articulating members 68 may have an ovoid cross-sectional shape. In some instances, the articulating members 68 may have a rectilinear or substantially rectilinear cross-sectional shape. As an example, the articulating members 68 may be formed of a ribbon material.

Relative rotation between the first disk 64 and the second disk 66 may cause the articulating members 68 to move from the collapsed configuration to the expanded configuration. In some cases, the collapsed configuration may represent a free state of the expandable frame 62. The expandable frame 62 may be actively expanded by rotating the first disk 64 in a counter-clockwise direction relative to the second disk 66. Alternatively, the expandable frame 62 may be actively expanded by rotating the second disk 66 in a clockwise direction relative to the first disk 64. In some cases, the expanded configuration may represent a free state of the expandable frame 62. The expandable frame 62 may expand passively when not constrained from reaching its free state, for example.

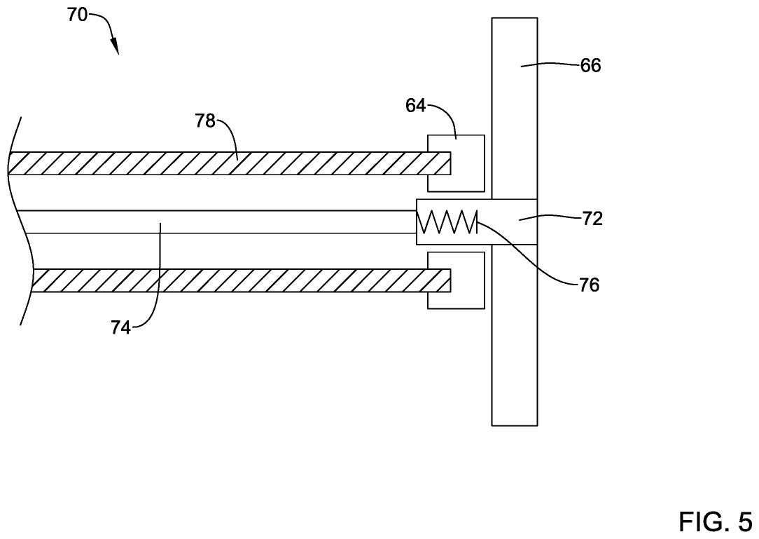

FIG. 5 is a schematic diagram showing the expandable frame 62 loaded into an illustrative delivery device 70. The delivery device 70 includes a tubular member 72 that is independent of a central member 74 and a pin 76 that attaches to the second disk 66, thereby allowing independent rotation of the first disk 64 and the second disk 66. An outer tubular member 78 is adapted to engage the first disk 64. Accordingly, rotation of the outer tubular member 78 causes rotation of the first disk 64 while rotation of the central member 74 causes rotation of the second disk 66.

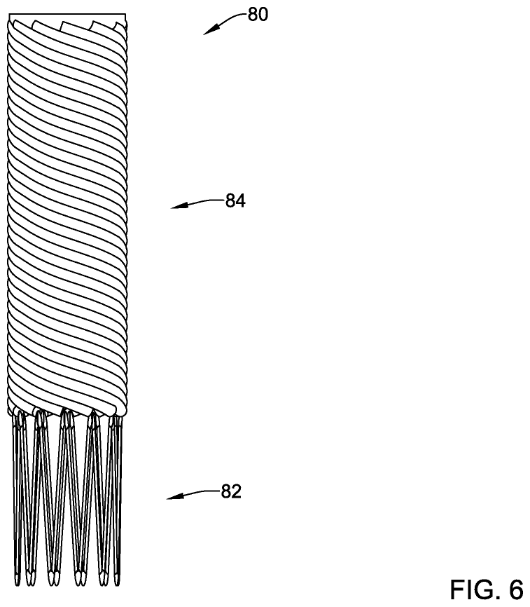

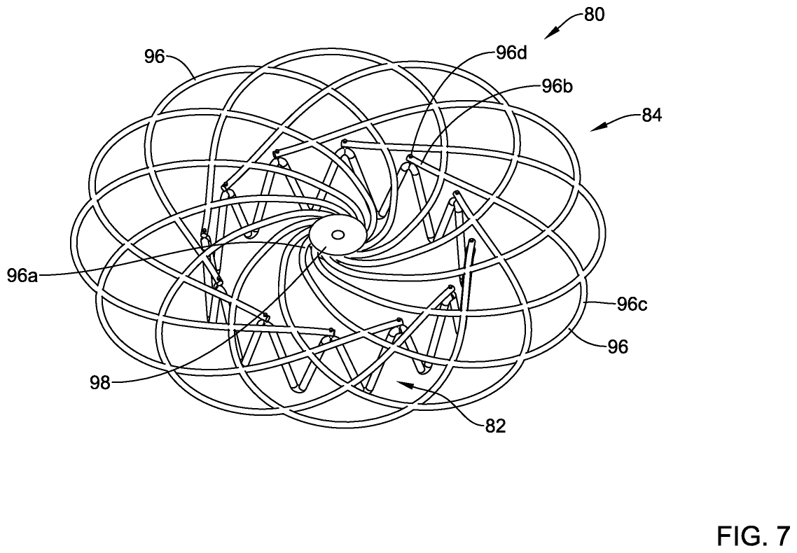

FIGS. 6 and 7 are perspective views of an illustrative expandable frame 80 that may be used in forming an LAAC device. In some cases, for example, an LAAC device could be formed simply by adding a covering (not shown) to the expandable frame 80. FIG. 6 shows the expandable frame 80 in a collapsed configuration while FIG. 7 shows the expandable frame 80 in an expanded configuration. The expandable frame 80 includes an expandable stent structure 82 and an expandable loop section 84. In some cases, the expandable stent structure 82 may be considered as being an example of the peripheral element 53 shown in FIGS. 2A, 2B and 2C. In some cases, the expandable stent structure 82 is adapted to be collapsed into a delivery configuration, as shown in FIG. 6.

As can be seen in FIG. 7, the expandable loop section 84 includes a number of articulating members 96, with each of the articulating members 96 having a first end 96a that is coupled to a center element 98, a second end 96b that is coupled to the expandable stent structure 82 and a loop portion 96c that extends between the first end 96a and the second end 96b. In some cases, the second end 96b of each of the articulating members 96 is pivotably secured relative to the expandable stent structure 82. As an example, the second end 96b of each of the articulating members 96 may be secured to the expandable stent structure 82 via a pin 96d that extends through the second end 96b and into the expandable stent structure 82. This is just an example. Rotation of the center element 98 relative to the expandable stent structure 82 causes the expandable loop section 84 to move into its expanded configuration, as shown in FIG. 7.

The articulating members 96 may be considered as being formed of a shape memory material that is adapted to have a remembered configuration. The articulating members 96 may be adapted to be deformed from the remembered configuration (such as collapsing the LAAC device into a delivery configuration), and to regain the remembered configuration once no longer constrained. As an example, the articulating members 68 may be formed of a shape memory metal such as Nitinol. As another example, the articulating members 96 may be formed of a shape memory polymer. The center element 98 may be formed of any suitable materials.

In some cases, the expandable stent structure 82 may be considered as being formed of a shape memory material that is adapted to have a remembered configuration. The expandable stent structure 82 may be adapted to be deformed from the remembered configuration (such as collapsing the LAAC device into a delivery configuration), and to regain the remembered configuration once no longer constrained. As an example, the expandable stent structure 82 may be formed of a shape memory metal such as Nitinol. As another example, the expandable stent structure 82 may be formed of a shape memory polymer.

In some cases, each of the articulating members 68 may have a circular or substantially circular cross-sectional shape. The articulating members 68 may be formed of a suitable wire, for example. In some cases, the articulating members 68 may have an ovoid cross-sectional shape. In some instances, the articulating members 68 may have a rectilinear or substantially rectilinear cross-sectional shape. As an example, the articulating members 68 may be formed of a ribbon material.

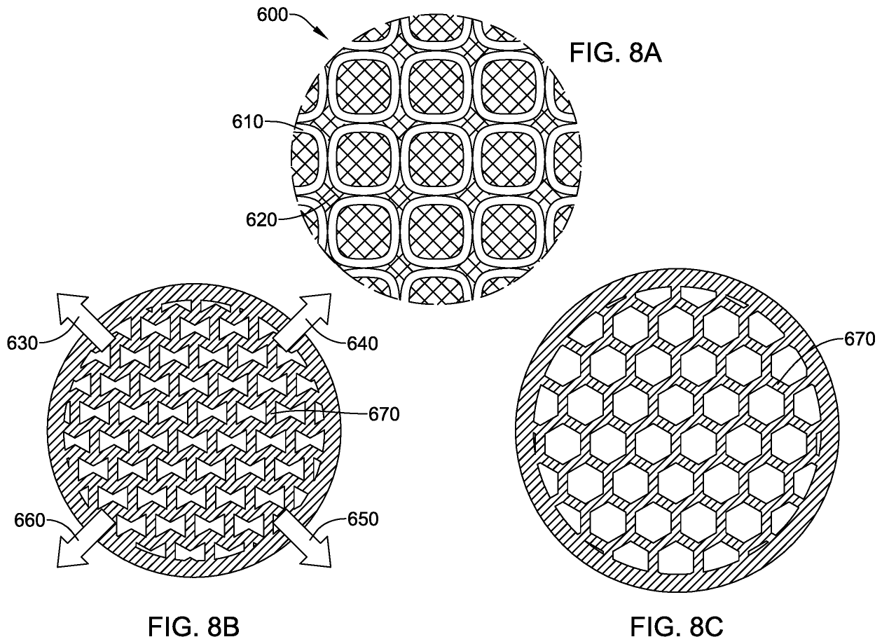

As will be appreciated, in some cases the covering (such as the occlusive element 120) that spans the expandable frame 110 may have to accommodate changes in the dimensions of the expandable frame 110. In other words, the covering may have to be able to stretch. FIGS. 8A, 8B and 8C together provide details of a covering 600 that may be used with the LAAC devices described herein. The covering 600 includes a webbing 610 that is made from a relatively thick fiber. A webbing 620 spans the distance between the relatively thick fibers forming the webbing 610 and is formed from relatively thin fibers. In some cases, the webbing 610 may be formed of fibers having an average diameter in a range of 5 to 10 μm and the webbing 620 may be formed of fibers having an average diameter in a range of 25 to 100 μm. In some cases, the webbing 610 is laid out in a honeycomb fashion, but this is not required in all cases.

In some cases, the webbing 610 may be formed of fibers that have a relatively large fraction of elastomer and a relatively smaller fraction of a second polymer such as but not limited to PET (polyethylene terephthalate). In some cases, the webbing 610 may be formed of fibers that are at least 50 percent elastomer and the webbing 620 may be formed of fibers that are at least 50 percent PET. In some cases, the webbing 610 may be formed of fibers that include about 70 percent elastomer and about 30 percent PET. In some cases, the webbing 620 may be formed of fibers that have a relatively large fraction of PET and a relatively smaller fraction of an elastomer. In some cases, the webbing 620 may be formed of fibers that include about 30 percent PET and about 70 percent elastomer. The elastomers used in the webbing 610 and the webbing 620 may include one or more of fluoroelastomers, polyurethane elastomers, Pebax, thermoplastic elastomers, copolyester elastomer, hydrophilic elastomers, polyamide 11 or polyether segments.

FIGS. 8B and 8C together illustrate how the covering 600 responds to applied forces. In particular, FIGS. 8B and 8C together show that tension applied in any direction, as indicated by the arrows 630, 640, 650 and 660, result in equal porosity. In FIG. 8C, the mesh 670 can be seen as having equal pore sizes. In some cases, the covering 600 may be considered as exhibiting auxetic properties. In some cases, the covering 600 may include materials such as urethane or nylon. In some cases, the covering 600 may also include radiopaque elements. In some cases, the covering 600 may be a fabric matrix that is formed in an auxetic pattern, such that stretching and compliance in a planar radial axis is equalized and distributed consistently regarding porosity of hemodynamic flow as well as hemostasis.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms. It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device, comprising:
an expandable frame comprising:
a peripheral element;
a central element rotatably disposed relative to the peripheral element;
a plurality of articulating members, each of the plurality of articulating members having a first end secured to the central element, a second end secured to the peripheral element and a loop portion extending therebetween; and
a covering disposed relative to the plurality of articulating members;
wherein relative rotation between the central element and the peripheral element in a first relative rotational direction causes the loop portions of each of the plurality of articulating members to extend radially outwardly beyond where the second end of each of the plurality of articulating members is secured to the peripheral element.

2. The implantable medical device of claim 1, wherein the central element comprises a first disk having a first diameter.

3. The implantable medical device of claim 1, wherein the peripheral element comprises a second disk having a second diameter greater than the first diameter.

4. The implantable medical device of claim 1, wherein the peripheral element comprises an expandable stent structure.

5. The implantable medical device of claim 1, wherein the second end of each of the plurality of articulating members is pivotably secured to the peripheral element.

6. The implantable medical device of claim 1, further comprising a plurality of pins pinning the second end of each of the plurality of articulating members to the peripheral element.

7. The implantable medical device of claim 1, wherein relative rotation of the central element between the peripheral element in a second relative rotational direction, opposing the first relative rotational direction, causes the loop portions of each of the plurality of articulating members to retract radially inwardly inside of where the second end of each of the plurality of articulating members is secured to the peripheral element.

8. The implantable medical device of claim 1, wherein the covering extends over the plurality of articulating members.

9. The implantable medical device of claim 1, wherein the plurality of articulating members are formed of a shape memory material.

10. The implantable medical device of claim 1, comprising an LAAC (left atrial appendage closure) device.

11. A left atrial appendage closure (LAAC) device, comprising:
a peripheral element;
a central element, where the central element and the peripheral element are adapted to rotate relative to each other;
a plurality of articulating members, each of the plurality of articulating members having a first end secured to the central element, a second end pivotably secured to the peripheral element and a loop portion extending therebetween; and
a covering disposed relative to the plurality of articulating members;
wherein relative rotation between the central element and the peripheral element in a first relative rotational direction causes the loop portions of each of the plurality of articulating members to extend radially outwardly beyond a periphery of the peripheral element.

12. The LAAC device of claim 11, wherein the covering extends over the plurality of articulating members.

13. The LAAC device of claim 11, wherein the central element comprises a first disk having a first diameter and the peripheral element comprises a second disk having a second diameter greater than the first diameter.

14. The LAAC device of claim 11, wherein the peripheral element comprises an expandable stent structure.

15. The LAAC device of claim 11, wherein the plurality of articulating members are formed of a shape memory material.

16. The LAAC device of claim 15, wherein the plurality of articulating members are adapted to have a remembered shape corresponding to a maximum value for the overall diameter of the LAAC device.

17. A left atrial appendage closure (LAAC) device, comprising:
an expandable frame moveable between a collapsed configuration and an expanded configuration, the expandable frame comprising:
a peripheral element;
a central element rotatably disposed relative to the peripheral element;
a plurality of articulating members, each of the plurality of articulating members having a first end secured to the central element, a second end secured to the peripheral element and a loop portion extending therebetween; and
a covering disposed relative to the plurality of articulating members;
wherein rotation of the central element relative to the peripheral element causes the loop portions of each of the plurality of articulating members to extend radially outwardly beyond a periphery of the peripheral element.

18. The LAAC device of claim 17, wherein the plurality of articulating members are formed of a shape memory material.

19. The LAAC device of claim 18, wherein the plurality of articulating members are adapted to have a remembered shape corresponding to the expanded configuration of the expandable frame.

20. The LAAC device of claim 17, wherein rotating the central member in a first direction causes the expandable frame to move towards the expanded configuration and rotating the central member in a second direction opposing the first direction causes the expandable frame to move towards the collapsed configuration.
